(19) United States
(12) Patent Application Publication
(10) Pub. No.: US 2014/0199267 A1
(43) Pub. Date: Jul. 17, 2014

(54) METHODS FOR AMELIORATING SYMPTOMS OR CONDITIONS CAUSED BY STRESS

(71) Applicant: Nestec SA, Vevey (CH)

(72) Inventors: Delphine Tissot-Favre, Gollion (CH); Yuanlong Pan, Chesterfield, MO (US)

(73) Assignee: Nestec SA, Vevey (CH)

(21) Appl. No.: 14/238,237

(22) PCT Filed: Aug. 7, 2012
(86) PCT No.: PCT/US2012/049825

§ 371 Date: Feb. 11, 2014

Related U.S. Application Data
(60) Provisional application No. 61/575,073, filed on Aug. 15, 2011.

(51) Int. Cl. A61K 31/164; A23K 1/18; A23L 1/30; A23K 1/16

(52) U.S. Cl. CPC A61K 31/164; A23K 1/164; A23K 1/1846; A23L 1/3008
USPC 424/93.1; 564/208; 514/627; 424/780; 426/648; 426/2

(57) ABSTRACT

The invention provides methods for using fatty acid alkanolamides for ameliorating one or more symptoms or conditions caused by stress, e.g., loss of appetite, or for maintaining and enhancing nutrient and caloric intake by an animal under stress or that has experienced stress. Generally, the fatty acid alkanolamides in amounts of from about 0.1 to about 1500 mg/kg/day before, during or after stress, preferably on a regular or daily basis as a component of a food composition or as a dietary supplement.

### CROSS REFERENCE TO RELATED APPLICATIONS

This application claims priority to U.S. Provisional Application Ser. No. 61/575,073 filed Aug. 15, 2011, the disclosure of which is incorporated herein by this reference.

### BACKGROUND OF THE INVENTION

1. Field of the Invention

The invention relates generally to methods for ameliorating symptoms or conditions caused by stress and particularly to methods for using fatty acid alkanolamides for ameliorating symptoms or conditions caused by stress.

2. Description of Related Art

Fatty acid alkanolamides are compounds containing a fatty acid moiety linked to alkanolamine. Fatty acid ethanolamides are compounds containing a fatty acid moiety linked to ethanolamine. These compounds are part of a family of lipids that are generally found in plant and animal tissues. Numerous fatty acid alkanolamides and fatty acid ethanolamides are known in the art, e.g., the compounds disclosed in U.S. Pat. No. 6,911,474 and WO05115370A2. Two common fatty acid ethanolamides are anandamide (N-arachidonoyl-ethanolamine) and N-oleoyl-ethanolamide.

U.S. Pat. No. 6,911,474, U.S. Pat. No. 6,911,474, U.S. Pat. No. 7,423,066, and US2005187254A1 disclose using fatty acid alkanolamides and their homologs and analogs to reduce body fat, reduce food consumption, and modulate lipid metabolism. WO08040756A1 discloses the use of anandamide for the manufacture of a nutraceutical for oral intake preferably a medicament for reducing appetite, giving a satiety effect, preventing or reducing inflammatory bowel disease, or preventing or reducing irritable bowel syndrome. WO2010019211A1 discloses compositions and methods for influencing satiety, lipid metabolism, and fat utilization. WO2009142713A1 discloses methods for reducing lipid absorption by an animal. However, the references do not disclose that such compounds affect symptoms or conditions caused by stress in an animal.

Stress has many definitions known to skilled artisans. Generally, stress is an animal's reaction to change that requires a physical, mental, or emotional adjustment or response. In some situations, stress causes the release of various neurochemicals and hormones that prepare an animal for action, e.g., fight or flight. Absent any action, the stress response can cause health problems for the animal. Prolonged, uninterrupted, unexpected, and unmanageable stresses are the most harmful types of stress.

There are known methods for affecting stress and the symptoms and conditions caused by stress. Drugs such as drugs that reduce depression can be used to affect stress and its associated symptoms and conditions. Anti-depressants such as Prozac®, Deroxat® and Zoloft® or anxiolytics such as Xanax®, Temesta®, Lexomil®, and Valium® are often prescribed for treating stress and affecting and the symptoms and conditions caused by stress. These methods, however, are often accompanied by one or more adverse side effects. Mediation, relaxation, hypnosis, exercise, counseling, and nutrition, are known methods for affecting stress and the symptoms and conditions caused by stress.

While these methods are arguably effective for affecting stress and some of its associated symptoms and conditions, there is a need for new methods for ameliorating the symptoms and conditions caused by stress, particularly unintrusive and convenient methods with minimal adverse side effects.

### SUMMARY OF THE INVENTION

It is, therefore, an object of the present invention to provide methods for ameliorating symptoms or conditions in an animal caused by stress.

It is another object of the present invention to provide methods for maintaining and enhancing nutrient and caloric intake by an animal under stress or that has experienced stress.

It is another object of the invention to provide compositions suitable for ameliorating symptoms or conditions in an animal caused by stress or for maintaining and enhancing nutrient and caloric intake by an animal under stress or that has experienced stress.

It is a further object of the invention to provide kits suitable for ameliorating symptoms or conditions in an animal caused by stress or for maintaining and enhancing nutrient and caloric intake by an animal under stress or that has experienced stress.

One or more of these or other objects are achieved using novel methods for ameliorating one or more symptoms or conditions in an animal caused by stress. The methods comprise administering one or more fatty acid alkanolamides to an animal in an amount effective for ameliorating one or more symptoms or conditions caused by stress or for maintaining and enhancing nutrient and caloric intake by an animal under stress or that has experienced stress.

Other and further objects, features, and advantages of the present invention will be readily apparent to those skilled in the art.

### DETAILED DESCRIPTION OF THE INVENTION

### Definitions

The term “N-oleoyl-ethanolamide” means N-oleoyl-ethanolamide and its analogs, homologs, precursors, prodrugs, or combination thereof that have an affect on symptoms or conditions caused by stress.

The term “fatty acid ethanolamides” means one or more fatty acid ethanolamides and their analogs, homologs, precursors, prodrugs, or combination thereof that have an affect on symptoms or conditions caused by stress, including N-oleoyl-ethanolamide.

The term “fatty acid alkanolamides” means one or more fatty acid alkanolamides and their analogs, homologs, precursors, prodrugs, or combination thereof that have an affect on symptoms or conditions caused by stress, including fatty acid ethanolamides and N-oleoyl-ethanolamide.

The term “animal” means an animal susceptible to or suffering from one or more symptoms or conditions caused by stress, including human, avian, bovine, canine, equine, feline, hicrine, lupine, murine, ovine, or porcine animals.

The term “companion animal” means domesticated animals such as cats, dogs, rabbits, guinea pigs, ferrets, hamsters, mice, gerbils, horses, cows, goats, sheep, donkeys, pigs, and the like.

The term “health or wellness of an animal” means the complete physical, mental, and social well-being of the animal, not merely the absence of disease or infirmity.

The term “quality of life” means the ability to enjoy normal life activities.

The term “extending the prime” means extending the number of years an animal lives a healthy life and not just extending the number of years an animal lives, e.g., an animal would be healthy in the prime of its life for a relatively longer time.

The term “food” or “food composition” means a product or composition that is intended for ingestion by an animal and that provides nutrition to the animal.

The term “mg/kg/day” means milligrams per kilogram of body weight per day.

The term “mg/day” means milligrams per day.

The term “or” is used herein as an inclusive term meaning that one can either choose between alternatives or choose all of them, e.g., administering “before or after” means that administration can occur before an event, after an event, or before the event and after the event.

The term “dietary supplement” means a product that is intended to be ingested in addition to a normal animal diet. Dietary supplements may be in any form, e.g., solid, liquid, gel, tablet, capsule, powder, and the like. Preferably they are provided in convenient dosage forms, e.g., in sachets. Dietary supplements can be provided in bulk consumer packages such as bulk powders, liquids, gels, or oils. Similarly such supplements can be provided in bulk quantities to be included in other food items such as snacks, treats, supplement bars, beverages, and the like.

The term “stress control agent” means any method, compound, composition, or drug useful for ameliorating one or more symptoms or conditions in an animal caused by stress, other than the fatty acid alkanolamides of the present invention. Such agents include, but are not limited to, drugs such as anti-depressants and methods such as mediation, relaxation, hypnosis, exercise, counseling, and the like.

The term “in conjunction” means that fatty acid alkanolamides, stress control agents, or other compounds or compositions of the present invention are administered to an animal (1) together in a food composition, dietary supplement, or other composition; (2) separately at the same or different frequency using the same or different administration routes at about the same time or periodically; or (3) for methods, the methods are conducted and the same time that the fatty acid alkanolamides, stress control agents, or other compounds or compositions are administered to an animal. “Periodically” means that a stress control agent or other composition is administered on a dosage schedule acceptable for a specific agent or composition. “About the same time” generally means that the fatty acid alkanolamides, stress control agents, or other compositions are administered at the same time or within about 24 hours of each other. “In conjunction” specifically includes administration schemes wherein a stress control agent or other composition is administered for a prescribed period and the fatty acid alkanolamides are administered indefinitely, e.g., in a food composition.

The term “regular basis” means at least monthly dosing with dietary formulations of the present invention and more preferably weekly dosing. More frequent dosing or consumption, such as twice or three times weekly, is preferred in certain embodiments. Still more preferred are regimens that comprise at least once daily consumption, e.g., when the compounds of the invention are a component of a food composition that is consumed at least once daily.

The term “before stress” means any period before the beginning of stress, preferably a period of 6 months before the beginning of stress, more preferably a period of 4 months before the beginning of stress, most preferably a period of 2 months before the beginning of stress. In various embodiments, the period before the beginning of stress is 1 month, 2 weeks, one week, or one day.

The term “after stress” means any period before the beginning of stress, preferably a period of 6 months before the beginning of stress, more preferably a period of 4 months before the beginning of stress, most preferably a period of 2 months before the beginning of stress. In various embodiments, the period before the beginning of stress is 1 month, 2 weeks, one week, or one day.

The term “during stress” means any period from the beginning of stress until the end of stress.

The term “single package” means that the components of a kit are physically associated in or with one or more containers and considered a unit for manufacture, distribution, sale, or use. Containers include, but are not limited to, bags, boxes, cartons, bottles, packages such as shrink wrap packages, stapled or otherwise affixed components, or combinations thereof. A single package may be containers of individual fatty acid alkanolamides and food compositions physically associated such that they are considered a unit for manufacture, distribution, sale, or use.

The term “virtual package” means that the components of a kit are associated by directions on one or more physical or virtual kit components instructing the user how to obtain the other components, e.g., in a bag or other container containing one component and directions instructing the user to go to a website, contact a recorded message or a fax-back service, view a visual message, or contact a caregiver or instructor to obtain instructions on how to use the kit or safety or technical information about one or more components of a kit.

All percentages expressed herein are by weight of the total weight of the composition unless expressed otherwise.

As used herein, ranges are used herein in shorthand, so as to avoid having to list and describe each and every value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “a supplement”, “a method”, or “a food” includes a plurality of such “supplements”, “methods”, or “foods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. Similarly, the term “examples,” particularly when followed by a listing of terms, is merely exemplary and illustrative and should not be deemed to be exclusive or comprehensive.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, the preferred compositions, methods, articles of manufacture, or other means or materials are described herein.

All patents, patent applications, publications, technical or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved.

### The Invention

In one aspect, the invention provides methods for ameliorating one or more symptoms or conditions in animals caused by stress. The methods comprise administering one or more fatty acid alkanolamides to the animals in an amount effective for ameliorating one or more symptoms or conditions caused by stress. The invention is based upon the discovery that fatty acid alkanolamides such as N-oleoyl-ethanolamide are effective for ameliorating symptoms or conditions caused by stress and that the administration of fatty acid alkanolamides is an unintrusive and convenient method for ameliorating symptoms or conditions caused by stress while causing few if any adverse side effects.

In another aspect, the invention provides methods for maintaining and enhancing nutrient and caloric intake by an animal under stress or that has experienced stress. The methods comprise administering one or more fatty acid alkanolamides to the animals in an amount effective for maintaining and enhancing nutrient and caloric intake by the animals before, during, or after stress. The invention is based upon the discovery that fatty acid alkanolamides such as N-oleoyl-ethanolamide are effective for maintaining and enhancing nutrient and caloric intake by an animal when the animal is under stress, e.g., stress caused by social, environmental, or other conditions. The fatty acid alkanolamides can be administered to the animals before stress or during stress. The invention is particularly useful when an animal under stress reduces its nutrient and caloric intake. Administering the compounds of the invention enhance nutrient and caloric intake to help ensure that the animal is getting need nutrients and calories. If needed, the fatty acid alkanolamides can be administered to the animals after stress, e.g., to ensure that the animal continues to get sufficient nutrients and calories to be healthy. The methods are unintrusive and convenient while ensuring that an animal under stress gets needed nutrient and calories necessary for good health and wellness and to ensure a good quality of life.

The fatty acid alkanolamides are administered to the animals at any time suitable for ameliorating one or more symptoms or conditions caused by stress or for maintaining and enhancing nutrient and caloric intake by an animal under stress or that has experienced stress. In various embodiments, the fatty acid alkanolamides are administered to the animals before, during, or after stress. When stress is anticipated or has occurred, fatty acid alkanolamides can be administered for a specific period before or after stress. For example, fatty acid alkanolamides can be administered to animals for any period before an anticipated stress, e.g., 180, 90, 60, 45, 30, 14, 7, or 3 days before stress is anticipated. Similarly, fatty acid alkanolamides can be administered to animals for any period after stress, e.g., 180, 90, 60, 45, 30, 14, 7, or 3 days after stress. In addition, fatty acid alkanolamides can be administered to animals during stress, e.g., for the entire period during stress or for any portion of the period during stress. In a preferred embodiment, the fatty acid alkanolamides are administered to the animals throughout its life, preferably as a dietary supplement of a component of a food composition. In preferred embodiments, the fatty acid alkanolamides are administered to the animals on a regular basis, preferably a daily basis.

In one embodiment, the fatty acid alkanolamides are fatty acid ethanolamides. In another, the fatty acid alkanolamides are N-acyl fatty acid ethanolamides. In a further, the fatty acid alkanolamide is N-oleoyl-ethanolamide. In another, the fatty acid alkanolamide is (Z)-(R)-9-octadecenamide, N-(2-hydroxyethyl,1-methyl).

In various embodiments, the fatty acid alkanolamides are hydrolysis-resistant fatty acid alkanolamide analogs, hydrolysis-resistant fatty acid ethanolamide analogs, or hydrolysis-resistant N-oleoyl-ethanolamide analogs such as (Z)-(R)-9-octadecenamide, N-(2-hydroxyethyl,1-methyl).

Fatty acid alkanolamides are administered to an animal in any amount effective for ameliorating one or more symptoms or conditions caused by stress. Generally, fatty acid alkanolamides are administered to an animal in amounts of from about 0.1 to about 1500 mg/kg/day, preferably from about 1 to about 1000 mg/kg/day, most preferably from about 5 to about 500 mg/kg/day. Amounts of about 100 mg/kg/day are preferred. In various embodiments, the fatty acid alkanolamides are administered to the animal in amounts of from about 1 to about 5000 mg/day, preferably from about 5 to about 3000 mg/day, more preferably from about 10 to about 1000 mg/day.

Fatty acid alkanolamides can be administered to the animal in any suitable form using any suitable administration route. For example, fatty acid alkanolamides can be administered in a fatty acid alkanolamides composition, in a food composition, in a dietary supplement, in a pharmaceutical composition, in a nutraceutical composition, or as a medicament. Similarly, fatty acid alkanolamides can be administered using any suitable administration route, e.g., orally. Preferably, fatty acid alkanolamides are administered orally to an animal as a dietary supplement or as an ingredient in a food composition.

In a preferred embodiment, fatty acid alkanolamides are administered to an animal as an ingredient in a food composition suitable for consumption by an animal, including humans and companion animals such as dogs and cats. Such compositions include complete foods intended to supply the necessary dietary requirements for an animal or food supplements such as animal treats.

In various embodiments, food compositions such as pet food compositions or pet treat compositions comprise from about 15% to about 50% crude protein. The crude protein material may comprise vegetable proteins such as soybean meal, soy protein concentrate, corn gluten meal, wheat gluten, cottonseed, and peanut meal, or animal proteins such as casein, albumin, and meat protein. Examples of meat protein useful herein include pork, lamb, equine, poultry, fish, and mixtures thereof.

The food compositions may further comprise from about 5% to about 40% fat, generally with about 5 to 50% of energy represented by lipids. Examples of suitable fats include animal fats and vegetable fats. Preferably the fat source is an animal fat source such as tallow. Vegetable oils such as corn oil, sunflower oil, safflower oil, rape seed oil, soy bean oil, olive oil and other oils rich in monounsaturated and polyunsaturated fatty acids, may also be used.

The food compositions may further comprise from about 15% to about 60% carbohydrate. Examples of suitable carbohydrates include grains or cereals such as rice, corn, milo, sorghum, alfalfa, barley, soybeans, canola, oats, wheat, and mixtures thereof. The compositions may also optionally comprise other materials such as dried whey and other dairy by-products.

The moisture content for such food compositions varies depending on the nature of the food composition. The food compositions may be dry compositions (e.g., kibble), semi-moist compositions, wet compositions, or any mixture thereof. In a preferred embodiment, the composition is a complete and nutritionally balanced pet food. In this embodiment, the pet food may be a “wet food”, “dry food”, or food of “intermediate moisture” content. “Wet food” describes pet foods that are typically sold in cans or foil bags and has a moisture content typically in the range of about 70% to about 90%. “Dry food” describes pet food that is of a similar composition to wet food but contains a limited moisture content typically in the range of about 5% to about 15% or 20% (typically in the form or small biscuit-like kibbles). In one preferred embodiment, the compositions have moisture content from about 5% to about 20%. Dry foods include a variety of foods of various moisture contents, such that they are relatively shelf-stable and resistant to microbial or fungal deterioration or contamination. Also preferred are dry food compositions that are extruded foods such as pet foods or snack foods for either humans or companion animals.

The food compositions may also comprise one or more fiber sources. The term “fiber” includes all sources of “bulk” in the food whether digestible or indigestible, soluble or insoluble, fermentable or nonfermentable. Preferred fibers are from plant sources such as marine plants but microbial sources of fiber may also be used. A variety of soluble or insoluble fibers may be utilized, as will be known to those of ordinary skill in the art. The fiber source can be beet pulp (from sugar beet), gum arabic, gum talha, psyllium, rice bran, carob bean gum, citrus pulp, pectin, fructooligosaccharide, short chain oligofructose, mannanoligofructose, soy fiber, arabinogalactan, galactooligosaccharide, arabinoxylan, or mixtures thereof.

Alternatively, the fiber source can be a fermentable fiber. Fermentable fiber has previously been described to provide a benefit to the immune system of a companion animal. Fermentable fiber or other compositions known to skilled artisans that provide a prebiotic to enhance the growth of probiotics within the intestine may also be incorporated into the composition to aid in the enhancement of the benefit provided by the present invention to the immune system of an animal.

In some embodiments, the ash content of the food composition ranges from less than 1% to about 15%, preferably from about 5% to about 10%.

In a preferred embodiment, the composition is a food composition comprising fatty acid alkanolamides and from about 15% to about 50% protein, from about 5% to about 40% fat, from about 5% to about 10% ash content, and having a moisture content of about 5% to about 20%. In other embodiments, the food composition further comprises prebiotics or probiotics as described herein. In other embodiments, the food composition further comprises stress control agents as described herein.

When administered in a food composition, fatty acid alkanolamides comprises from about 1 to about 40% of the food composition, preferably from about 3 to about 30%, more preferably from about 5 to about 20%. In various embodiments, food compositions comprise about 1%, 2%, 4%, 6%, 8%, 10%, 12%, 14%, 16%, 18%, 20%, 22%, 24%, 26%, 28%, 30%, 32%, 34%, 36%, 38%, or 40%.

In another embodiment, fatty acid alkanolamides are administered to an animal in a dietary supplement. The dietary supplement can have any suitable form such as a gravy, drinking water, beverage, yogurt, powder, granule, paste, suspension, chew, morsel, treat, snack, pellet, pill, capsule, tablet, sachet, or any other suitable delivery form. The dietary supplement can comprise fatty acid alkanolamides and optional compounds such as vitamins, preservatives, probiotics, prebiotics, and antioxidants. This permits the supplement to be administered to the animal in small amounts, or in the alternative, can be diluted before administration to an animal. The dietary supplement may require admixing with a food composition or with water or other diluent prior to administration to the animal. When administered in a dietary supplement, fatty acid alkanolamides comprises from about 1 to about 80% of the supplement, preferably from about 3 to about 50%, more preferably from about 5 to about 40%.

In another embodiment, fatty acid alkanolamides are administered to an animal in a pharmaceutical or nutraceutical composition. The pharmaceutical composition comprises fatty acid alkanolamides and one or more pharmaceutically or nutraceutically acceptable carriers, diluents, or excipients. Generally, pharmaceutical compositions are prepared by admixing a compound or composition with excipients, buffers, binders, plasticizers, colorants, diluents, compressing agents, lubricants, flavorants, moistening agents, and the like, including other ingredients known to skilled artisans to be useful for producing pharmaceuticals and formulating compositions that are suitable for administration to an animal as pharmaceuticals. When administered in a pharmaceutical or nutraceutical composition, fatty acid alkanolamides comprises from about 1 to about 90% of the composition, preferably from about 3 to about 60%, more preferably from about 5 to about 50%.

In one embodiment, the fatty acid alkanolamides are administered in conjunction with one or more stress control agents. In another, the fatty acid alkanolamides are administered in conjunction with one or more prebiotics or probiotics. In a further, the fatty acid alkanolamides are administered in conjunction with one or more stress control agents and one or more prebiotics or probiotics.

The fatty acid alkanolamides are administered to any animal, preferably humans and companion animals such as canines and felines, preferably dogs and cats.

In various embodiments, the symptom or condition caused by stress is one or more of decrease in appetite, weight loss, alterations of the immune system, e.g., a decrease in the immune system (humoral or cell-mediated immunity), post-traumatic stress disorder, alteration of blood pressure, e.g., an increase in blood pressure, delay in recovery from stress, premature aging, gastrointestinal disturbance, e.g., stress induced diarrhea or constipation, incontinence, increase in appetite, weight gain, insomnia, headache, pain, dermatology conditions, e.g., skin conditions such as eczema or acne, muscle tension, infertility, muscle aches, fatigue, memory problems, inability to concentrate, poor judgment, worrying, moodiness, irritability, temper, agitation, inability to relax, depression, nausea, dizziness, loss of libido, insomnia, unwanted habits, e.g., drug use, alcohol consumption, smoking, nail biting, and the like.

In various embodiments, the stress is caused by one or more of undergoing anesthesia, major life change, lack of sleep, illness, surgery, chronic pain, bullying, relationship problems, e.g., divorce, financial difficulty, noise, traffic conditions, pollution, crime, e.g., victim of crime or fear of crime, unrealistic expectations, pessimism, or combinations thereof.

In one embodiment, the stress is caused by surgery, either anticipation or occurrence of surgery. In another embodiment, the stress is cause by undergoing anesthesia. In these embodiments, the fatty acid alkanolamides are administered before surgery or undergoing anesthesia or after surgery or undergoing anesthesia. The fatty acid alkanolamides ameliorate the adverse symptoms and conditions caused by surgery or undergoing anesthesia, particularly the conditions that adversely affect appetite or food intake. By ameliorating these conditions, the animal that has had surgery or undergone anesthesia will recover faster than an animal that has not been administered fatty acid alkanolamides. Generally, animals administered fatty acid alkanolamides benefits from better nutrition because the animal consumes the proper amounts and types of desired foods.

In still another aspect, the invention provides compositions comprising one or more fatty acid alkanolamides in an amount effective for ameliorating one or more symptoms or conditions in an animal caused by stress or effective for maintaining and enhancing nutrient and caloric intake by an animal under stress or that has experienced stress. The compositions contain fatty acid alkanolamides in any amount suitable for ameliorating one or more symptoms or conditions caused by stress or for maintaining and enhancing nutrient and caloric intake by an animal under stress or that has experienced stress. Generally, the compositions contain fatty acid alkanolamides in amounts sufficient to administer fatty acid alkanolamides to an animal in amounts of from about 0.1 to about 1500 mg/kg/day, preferably from about 1 to about 1000 mg/kg/day, most preferably from about 5 to about 500 mg/kg/day when the compositions are administered as anticipated or recommended for a particular composition. Typically, fatty acid alkanolamides comprise from about 1 to about 80% of the composition, preferably from about 3 to about 50%, more preferably from about 5 to about 30%. In various embodiments, food compositions comprise about 1%, 2%, 4%, 6%, 8%, 10%, 12%, 14%, 16%, 18%, 20%, 22%, 24%, 26%, 28%, 30%, 32%, 34%, 36%, 38%, 40%, 45%, 50%, 55%, 60%, 70%, or 80%.

In preferred embodiments, the compositions further comprise one or more stress control agents. Any stress control agent can be used, e.g., urinary antispasmodics, antipsychotics, prazosin, sedatives (also referred to as tranquilizers) such as benzodiazepines and barbiturates, antidepressants such as selective serotonin reuptake inhibitors (SSRIs), beta-blockers such as propranolol and atenolol, and nutritional supplements such as Kava, Passion flower, Valerian, 5-HTP and various herbs. Many such stress control agents are known to skilled artisans.

Preferably, the fatty acid alkanolamides are fatty acid ethanolamides or N-acyl fatty acid ethanolamide. Most preferably, the fatty acid alkanolamides are selected from the group consisting of N-oleoyl-ethanolamide and (Z)-(R)-9-octadecenamide, N-(2-hydroxyethyl,1-methyl).

Fatty acid alkanolamides compositions such as food, dietary, pharmaceutical, and other compositions may further comprise one or more substances such as vitamins, minerals, probiotics, prebiotics, salts, and functional additives such as palatants, colorants, emulsifiers, and antimicrobial or other preservatives. Minerals that may be useful in such compositions include, for example, calcium, phosphorous, potassium, sodium, iron, chloride, boron, copper, zinc, magnesium, manganese, iodine, selenium, and the like. Examples of additional vitamins useful herein include such fat soluble vitamins as A, D, E, and K. Inulin, amino acids, enzymes, coenzymes, and the like may be useful to include in various embodiments.

In various embodiments, the compositions further comprise at least one of (1) one or more probiotics; (2) one or more inactivated probiotics; (3) one or more components of inactivated probiotics that promote health benefits similar to or the same as the probiotics, e.g., proteins, lipids, glycoproteins, and the like; (4) one or more prebiotics; and (5) combinations thereof. The probiotics or their components can be integrated into the compositions (e.g., uniformly or non-uniformly distributed in the compositions) or applied to the compositions (e.g., topically applied with or without a carrier). Such methods are known to skilled artisans, e.g., U.S. Pat. No. 5,968,569 and related patents. In preferred embodiments, the compositions are food compositions as described herein.

Typical probiotics include, but are not limited to, probiotic strains selected from Lactobacilli, Bifidobacteria, or Enterococci, e.g., Lactobacillus reuteii, Lactobacillus acidophilus, Lactobacillus animalis, Lactobacillus ruminis, Lactobacillus johnsonii, Lactobacillus casei, Lactobacillus paracasei, Lactobacillus rhamnosus, Lactobacillus fermentum, and Bifidobacterium sp., Enterococcus faecium and Enterococcus sp. In some embodiments, the probiotic strain is selected from the group consisting of Lactobacillus reuteri (NCC2581; CNCM I-2448), Lactobacillus reuteri (NCC2592; CNCM I-2450), Lactobacillus rhamnosus (NCC2583; CNCM I-2449), Lactobacillus reuteri (NCC2603; CNCM I-2451), Lactobacillus reuteri (NCC2613; CNCM I-2452), Lactobacillus acidophilus (NCC2628; CNCM I-2453), Bifidobacterium adolescentis (e.g., NCC2627), Bifidobacterium sp. NCC2657 or Enterococcus faecium SF68 (NCIMB 10415). The compositions contain probiotics in amounts sufficient to supply from about 10⁴ to about 10¹² cfu/animal/day, preferably from 10⁵ to about 10¹¹ cfu/animal/day, most preferably from 10⁷ to 10¹⁰ cfu/animal/day. When the probiotics are killed or inactivated, the amount of killed or inactivated probiotics or their components should produce a similar beneficial effect as the live microorganisms. Many such probiotics and their benefits are known to skilled artisans, e.g., EP1213970B1, EP1143806B1, U.S. Pat. No. 7,189,390, EP1482811B1, EP1296565B1, and U.S. Pat. No. 6,929,793. In a preferred embodiment, the probiotic is Enterococcus faecium SF68 (NCIMB 10415). In one embodiment, the probiotics are encapsulated in a carrier using methods and materials known to skilled artisans.

As stated, the compositions, e.g., food compositions, may contain one or more prebiotics, e.g., fructo-oligosaccharides, gluco-oligosaccharides, galacto-oligosaccharides, isomalto-oligosaccharides, xylo-oligosaccharides, soybean oligosaccharides, lactosucrose, lactulose, and isomaltulose. Fructo-oligosaccharides are found naturally in many foods such as wheat, onions, bananas, honey, garlic, and leeks. In one embodiment, the prebiotic is chicory root, chicory root extract, inulin, or combinations thereof. Generally, prebiotics are administered in amounts sufficient to positively stimulate the healthy microflora in the gut and cause these “good” bacteria to reproduce. Typical amounts are from about one to about 10 grams per serving or from about 5% to about 40% of the recommended daily dietary fiber for an animal. The amount of prebiotic can be determined by the skilled artisan based upon (1) the type and nature of the prebiotic and the type and nature of the desired composition and (2) the type and nature of the animal that will consume the composition, e.g., the animal's age, weight, general health, sex, gut microflora status (including presence of harmful bacteria), and diet.

The probiotics and prebiotics can be made part of the composition by any suitable means. Generally, the agents are mixed with the composition or applied to the surface of the composition, e.g., by sprinkling or spraying. When the agents are part of a kit, the agents can be admixed with other materials or in their own package. Typically, the food composition contains from about 0.1 to about 10% prebiotic, preferably from about 0.3 to about 7%, most preferably from about 0.5 to 5%, on a dry matter basis. The prebiotics can be integrated into the compositions using methods known to skilled artisans, e.g., U.S. Pat. No. 5,952,033.

A skilled artisan can determine the appropriate amount of fatty acid alkanolamides, food ingredients, vitamins, minerals, probiotics, prebiotics, antioxidants, or other ingredients to be used to make a particular composition to be administered to a particular animal. Such artisan can consider the animal's species, age, size, weight, health, and the like in determining how best to formulate a particular composition comprising fatty acid alkanolamides and other ingredients. Other factors that may be considered include the type of composition (e.g., pet food composition versus dietary supplement), the desired dosage of each component, the average consumption of specific types of compositions by different animals (e.g., based on species, body weight, activity/energy demands, and the like), and the manufacturing requirements for the composition.

In a further aspect, the present invention provides kits suitable for ameliorating one or more symptoms or conditions caused by stress or suitable for maintaining and enhancing nutrient and caloric intake by an animal under stress or that has experienced stress by administering fatty acid alkanolamides to an animal. The kits comprise in separate containers in a single package or in separate containers in a virtual package, as appropriate for the kit component, one or more fatty acid alkanolamides and one or more of (1) one or more ingredients suitable for consumption by an animal, e.g., food ingredients or beverages; (2) one or more probiotics or prebiotics; (3) one or more stress control agents; (4) instructions for how to combine fatty acid alkanolamides and other kit components to produce a composition useful for ameliorating one or more symptoms or conditions caused by stress; (5) instructions for how to combine fatty acid alkanolamides and other kit components to produce a composition useful for maintaining and enhancing nutrient and caloric intake by an animal under stress or that has experienced stress; (6) instructions for how to use fatty acid alkanolamides and other components of the present invention such as stress control agents, particularly for the benefit of the animal; (7) a device for preparing or combining the kit components to produce a composition for administration to an animal such as a spoon or other application device; and (8) a device for administering the combined or prepared kit components to an animal such as a bowl or other container.

When the kit comprises a virtual package, the kit is limited to instructions in a virtual environment in combination with one or more physical kit components. The kit contains fatty acid alkanolamides and other components in amounts sufficient to ameliorate one or more symptoms or conditions caused by stress. Typically, fatty acid alkanolamides and the other suitable kit components are admixed just prior to consumption by an animal. The kits may contain the kit components in any of various combinations or mixtures. In one embodiment, the kit contains a packet containing fatty acid alkanolamides and a container of food for consumption by an animal. The kit may contain additional items such as a device for mixing fatty acid alkanolamides and other ingredients or a device for containing the admixture, e.g., a food bowl. In another embodiment, fatty acid alkanolamides are mixed with additional nutritional supplements such as vitamins and minerals that promote good health in an animal. The components are each provided in separate containers in a single package or in mixtures of various components in different packages. In preferred embodiments, the kits comprise fatty acid alkanolamides and one or more other ingredients suitable for consumption by an animal. Preferably such kits comprise instructions describing how to combine fatty acid alkanolamides with the other ingredients to form a food composition for consumption by the animal, generally by mixing fatty acid alkanolamides with the other ingredients or by applying fatty acid alkanolamides to the other ingredients, e.g., by sprinkling fatty acid alkanolamides on a food composition.

In another aspect, the invention provides a means for communicating information about or instructions for one or more of (1) using fatty acid alkanolamides for ameliorating one or more symptoms or conditions in an animal caused by stress; (2) using fatty acid alkanolamides for maintaining and enhancing nutrient and caloric intake by an animal under stress or that has experienced stress; (3) admixing fatty acid alkanolamides and other ingredients to produce a fatty acid alkanolamides composition such as a fatty acid alkanolamides food composition or fatty acid alkanolamides dietary supplement suitable for consumption by an animal; (4) using the kits of the present invention for the benefit of an animal, e.g., increasing or maintaining appetite after surgery or anesthesia or maintaining immune function; and (5) using fatty acid alkanolamides in conjunction with stress control agents for the benefit of the animal; and (6) administering fatty acid alkanolamides to an animal. The means comprises one or more of a physical or electronic document, digital storage media, optical storage media, audio presentation, audiovisual display, or visual display containing the information or instructions. Preferably, the means is selected from the group consisting of a displayed website, a visual display kiosk, a brochure, a product label, a package insert, an advertisement, a handout, a public announcement, an audiotape, a videotape, a DVD, a CD-ROM, a computer readable chip, a computer readable card, a computer readable disk, a USB device, a FireWire device, a computer memory, and any combination thereof.

In a further aspect, the invention provides methods for manufacturing a food composition comprising fatty acid alkanolamides and one or more other ingredients suitable for consumption by an animal, e.g., one or more of protein, fat, carbohydrate, fiber, vitamins, minerals, probiotics, prebiotics, stress control agents, and the like. The methods comprise admixing one or more ingredients suitable for consumption by an animal with fatty acid alkanolamides. Alternatively, the methods comprise applying fatty acid alkanolamides alone or in conjunction or combination with other ingredients onto the food composition, e.g., as a coating or topping. Fatty acid alkanolamides can be added at any time during the manufacture or processing of the food composition. The composition can be made according to any method suitable in the art. In other embodiments, the methods further comprise admixing one or more stress control agents with the ingredients and fatty acid alkanolamides. In one embodiment, the methods further comprise admixing or applying one or more stress control agents with the fatty acid alkanolamides and ingredients.

In another aspect, the invention provides packages useful for containing fatty acid alkanolamides. The packages comprise at least one material suitable for containing fatty acid alkanolamides and a label affixed to the material containing a word or words, picture, design, acronym, slogan, phrase, or other device, or combination thereof, that indicates that the package contains fatty acid alkanolamides, compounds or compositions useful for ameliorating one or more symptoms or conditions caused by stress, compounds or compositions useful for maintaining and enhancing nutrient and caloric intake by an animal under stress or that has experienced stress, or combination thereof. Typically, such device comprises the words “alleviates stress symptoms” or “contains compounds that combat conditions caused by stress” or “contains compounds useful for maintaining and enhancing nutrient and caloric intake by an animal under stress or that has experienced stress, or “contains compounds useful for maintaining and enhancing nutrient and caloric intake by an animal after surgery, or an equivalent expression printed on the material. Any package configuration and packaging material suitable for containing fatty acid alkanolamides are useful in the invention, e.g., a bag, box, sachet, bottle, can, pouch, and the like manufactured from paper, plastic, foil, metal, and the like. In preferred embodiments, the package further comprises one or more fatty acid alkanolamides. In various embodiments, the package further comprises at least one window that permit the package contents to be viewed without opening the package. In some embodiments, the window is a transparent portion of the packaging material.

In another aspect, the invention provides for use of fatty acid alkanolamides to prepare a medicament for (1) ameliorating one or more symptoms or conditions in animals caused by stress or (2) maintaining and enhancing nutrient and caloric intake by an animal under stress. Generally, medicaments are prepared by admixing a compound or composition, i.e., fatty acid alkanolamides or fatty acid ethanolamides, with excipients, buffers, binders, plasticizers, colorants, diluents, compressing agents, lubricants, flavorants, moistening agents, and other ingredients known to skilled artisans to be useful for producing medicaments and formulating medicaments that are suitable for administration to an animal.

In a further aspect, the invention provides methods for promoting the health or wellness of animals. The method comprises (1) identifying an animal whose health and wellness will be promoted by ameliorating one or more symptoms or conditions in animals caused by stress or by maintaining and enhancing nutrient and caloric intake by the animal during stress and (2) administering one or more fatty acid alkanolamides to the animal in an amount effective for ameliorating the symptoms or conditions caused by stress or by enhancing nutrient and caloric intake by the animal under stress, thereby promoting the health or wellness of the animal.

In another aspect, the invention provides methods for improving the quality of life for animals. The method comprises (1) identifying an animal whose quality of life will be improved by ameliorating one or more symptoms or conditions caused by stress or by enhancing nutrient and caloric intake by the animal under stress and (2) administering one or more fatty acid alkanolamides to the animal in an amount effective for ameliorating the symptoms or conditions caused by stress or for enhancing nutrient and caloric intake by the animal during stress, thereby increasing the quality of life for the animal.

In another aspect, the invention provides methods for extending the prime years of an animal's life. The methods comprise administering one or more fatty acid alkanolamides to the animal in an amount effective for ameliorating the symptoms or conditions caused by stress or by enhancing nutrient and caloric intake by the animal during stress, thereby extending the prime for the animal.

In a further aspect, the invention provides methods for ameliorating one or more symptoms or conditions caused by stress induced by anesthesia in an animal or for maintaining and enhancing nutrient and caloric intake by an animal under stress or that has experienced stress induced by anesthesia. The methods comprise administering a stress symptom or condition ameliorating amount or a nutrient and caloric intake maintaining or enhancing amount of one or more fatty acid alkanolamides to the animal before or after undergoing anesthesia. In one embodiment, the animal has undergone anesthesia as part of a surgical procedure. Symptoms or conditions ameliorated include a loss of appetite and a decrease of food intake.

In a further aspect, the invention provides methods for ameliorating one or more symptoms or conditions caused by stress resulting from surgery on an animal or for maintaining and enhancing nutrient and caloric intake by an animal under stress or that has experienced stress caused by surgery. The methods comprise administering a stress symptom or condition ameliorating amount or a nutrient and caloric intake maintaining or enhancing amount of one or more fatty acid alkanolamides to the animal before or after surgery. Symptoms or conditions ameliorated include a loss of appetite and a decrease of food intake.

In a further aspect, the invention provides methods for affecting one or more structures or functions of an animal susceptible to or under stress. The method comprises (1) identifying an animal susceptible to or under stress that could adversely affect one or more of the animal's structures or functions and (2) administering one or more fatty acid alkanolamides to the animal in an amount effective for affecting the animal's structure or function altered by the stress, e.g., partially of completely preventing a change on alteration to the animal's structure of function when the animal is under stress or having an affect that partially or completely restores the animal's structure of function to the status the structure or function was before the stress.

In various embodiments, the methods are effective for one or more of helping maintain cardiovascular function; helping maintain a healthy circulatory system; helping maintain beneficial intestinal flora; promoting relaxation; reducing frustration; reducing the normal symptomatology of premenstrual syndrome or menopause; ameliorating occasional heartburn; ameliorating occasional acid indigestion; ameliorating occasional simple nervous tension; reducing nervousness due to common every day overwork and fatigue; creating a relaxed feeling; facilitating calming down and relaxing; reducing fatigue; gently soothing away tension; reducing irritability; and reducing restlessness. These structures and functions are often adversely affected by stress and are positively affected by ameliorating one or more symptoms or conditions in an animal caused by stress.

### EXAMPLES

The invention can be further illustrated by the following examples, although it will be understood that these examples are included merely for purposes of illustration and are not intended to limit the scope of the invention unless otherwise specifically indicated.

### Example 1

Seventeen (17) adult Beagles with a percentage of body fat of less than 17.5% in male dogs and less than 20% in female dogs were selected. Based on minimum energy requirements (MER), body weight, and percent body fat, the dogs were randomly assigned into two groups. The control group (N=9) received a standard dry adult dog food (without any N-oleoyl-ethanolamide). The test group (N=9) received the selected dose of N-oleoyl-ethanolamide incorporated into the standard diet. The dogs were fed 25% more than their MER for 4 months.

At month 2 and 4, the dogs were anesthetized to cause stress and their body composition was measured by DEXA. The results are shown in Table 1.

TABLE 1
Effect of N-oleoyl-ethanolamide on the Maintenance
of Normal Food Intake after a Stress Event

| | | Control | N-oleoyl- |
|---|---|---|---|
| | | Diet | ethanolamide |
| | | (N = 9) | Diet (N = 9) |
| Percentage | Average over | 97 | 99 |
| of | 4 Months | | |
| Daily | Average over | 83.5 | 94 |
| Ration | the Week | | |
| Consumed | of the Stress Event | | |

| | | |
|---|---|---|
| Reduction in Food Intake | −13.5 | −5 |
| Stressful Event (%) | | |

TABLE 2
Effect of N-oleoyl-ethanolamide on the Acute Impact
of Stress on Food Intake on the Day of the Stressful Event

| | N-oleoyl- | |
|---|---|---|
| | ethanolamide Diet | Control Diet |
| Average Percentage of | 96 | 35 |
| Daily Ration Consumed | | |

Referring to Table 1 and Table 2, during the week of the DEXA-related anesthesia events, the dogs fed the N-oleoyl-ethanolamide-containing diet consumed daily 94% of their daily ration, compared to an average daily food intake of 99% over the 4 months. In contrast, the dogs fed the standard adult diet consumed 83.5% of their daily ration, compared to an average daily food intake of 97% over the 4 months. On an individual basis, in the group of dog fed the N-oleoyl-ethanolamide-containing diet, no dog had a high reduction of their food intake (from 60 to 100% reduction) on the day of anesthesia (month 2). In contrast, in the group of dog fed the standard adult diet, 6 dogs form the 9 dogs had a high reduction in food intake (from 60 to 100%) on the day of anesthesia (month 2). The impact on the food intake lasted on more than 3 weeks for some of the dogs.

In the specification, there have been disclosed typical preferred embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims, the invention may be practiced otherwise than as specifically described.

1. A composition comprising one or more fatty acid alkanolamides in an amount effective for ameliorating one or more symptoms or conditions in an animal caused by stress or effective for maintaining and enhancing nutrient and caloric intake by an animal under stress or that has experienced stress and one or more stress control agents.

2. The composition of claim 1, wherein the fatty acid alkanolamides comprise from about 1 to about 80% of the composition.

3. The composition of claim 1, further comprising at least one of (1) one or more probiotics; (2) one or more inactivated probiotics; (3) one or more components of inactivated probiotics that promote health benefits similar to or the same as the probiotics; (4) one or more prebiotics; or (5) combinations thereof.

4. The composition of claim 1, wherein the fatty acid alkanolamide is a fatty acid ethanolamide.

5. The composition of claim 1, wherein the fatty acid alkanolamide is (Z)-(R)-9-octadecenamide, N-(2-hydroxyethyl,1-methyl).

6. The composition of claim 1, wherein the fatty acid alkanolamide is a hydrolysis-resistant fatty acid alkanolamide analog.

7. The composition of claim 1, containing fatty acid alkanolamides in amounts sufficient to administer fatty acid alkanolamides to an animal in amounts of from about 0.1 to about 1500 mg/kg/day.

8. The composition of claim 1, wherein the composition is a food composition.

9. The composition of claim 8, wherein the food composition is a companion animal food composition.

10. The composition of claim 8, containing fatty acid alkanolamides in amounts sufficient to administer fatty acid alkanolamides to an animal in amounts of from about 0.1 to about 1500 mg/kg/day.

11. The composition of claim 8, wherein the fatty acid alkanolamides comprise from about 1 to about 40% of the food composition.

12. A method for ameliorating one or more symptoms or conditions in an animal caused by stress comprising administering one or more fatty acid alkanolamides to the animal in an amount effective for ameliorating the symptoms or conditions.

13. The method of claim 12, wherein the fatty acid alkanolamides are administered before, during, or after stress.

14. The method of claim 12, wherein fatty acid alkanolamides are administered to the animal in a pharmaceutical or nutraceutical composition.

15. The method of claim 12, further comprising administering the fatty acid alkanolamides in conjunction with one or more stress control agents.

16. The method of claim 12, further comprising administering the fatty acid alkanolamides in conjunction with one or more prebiotics or probiotics.

17. The method of claim 12, wherein the animal is a human or a companion animal.

18. The method of claim 17, wherein the companion animal is a canine or a feline.

19. The method of claim 12, wherein the symptom or condition caused by stress is one or more of decrease in appetite, weight loss, alterations of the immune system, post-traumatic stress disorder, alteration of blood pressure, delay in recovery from stress, premature aging, gastrointestinal disturbance, incontinence, increase in appetite, weight gain, insomnia, headache, pain, dermatology conditions, muscle tension, infertility, muscle aches, fatigue, memory problems, inability to concentrate, poor judgment, worrying, moodiness, irritability, temper, agitation, inability to relax, depression, nausea, dizziness, loss of libido, insomnia, unwanted habits, or combinations thereof; wherein the stress is caused by one or more of undergoing anesthesia, major life change, lack of sleep, illness, surgery, chronic pain, bullying, relationship problems, financial difficulty, noise, traffic conditions, pollution, crime, unrealistic expectations, pessimism, or combinations thereof; wherein the stress is caused by surgery; or wherein the stress is caused by undergoing anesthesia.

20. A method for maintaining or enhancing nutrient and caloric intake by an animal under stress or that has experienced stress comprising administering one or more fatty acid alkanolamides to the animal in an amount effective for maintaining and enhancing nutrient and caloric intake by the animal before, during, or after stress.
